(19) United States
(12) Patent Application Publication
(10) Pub. No.: US 2012/0107393 A1
(43) Pub. Date: May 3, 2012

(54) Pellets for Delivery of Biologically Active Substances

(75) Inventors: Sonia Bedi, Plainsboro, NJ (US); Atul Shukla, Cordova, TN (US); Krishna Shukla, Cordova, TN (US)

(73) Assignee: UNIVERSITY OF TENNESSEE RESEARCH FOUNDATION, Knoxville, TN (US)

(21) Appl. No.: 13/279,646

(22) Filed: Oct. 24, 2011

Related U.S. Application Data
(60) Provisional application No. 61/456,008, filed on Oct. 29, 2010.

(51) Int. Cl. A61K 9/48; A61P 43/00; A61K 31/635; A61K 9/14; A61K 31/167

(52) U.S. Cl. 424/451; 424/490; 514/629; 514/158

(57) ABSTRACT

Pellets are made by combining a direct compression binder and a waxy spheronizing agent. The pellets may further contain additional pharmaceutical ingredients and/or a drug. The pellets may be compressed to form tablets or loaded into a capsule shell for oral administration, and the pellets may or may not have an additional coating.

This application claims the benefit of pending U.S. provisional patent application Ser. No. 61/456,008, which was filed on Oct. 29, 2010.

### FIELD OF THE INVENTION

The present application pertains to the field of solid dosage forms for administration of active pharmaceutical agents to an individual. In particular, the application pertains to the administration of active pharmaceutical agents by the administration of a dosage form containing a multiplicity of pellets containing the active pharmaceutical agent.

### BACKGROUND

Pellets are spherical multiparticulate dosage forms that are uniform in particle size and robust, and have a smooth surface for easy coating applications. Pellets have been of growing interest due to their multiparticulate nature as, when orally administered, they can easily pass through the pyloric sphincter and reach the key site of absorption in the intestine. Also, due to their narrow particle size distribution, they provide a uniform distribution of drug and a consistent coating thickness due to their smooth surface.

To date, microcrystalline cellulose (MCC) is the principle excipient that has been able to meet all the desired specifications of these pellets. In addition to MCC, various alternative excipients have been evaluated for pellet production, including chitosan, hydroxypropyl methyl cellulose (HPMC), cross-linked carboxymethyl cellulose sodium, carrageenan, and cellulose and starch derivatives. However, unlike MCC, none of these alternative materials has provided the same flexibility in formulation and processing during extrusion-spheronization as observed for MCC due to their inability to offer most of the inherent features of MCC including plastic deformation and brittleness. In addition, pellets prepared with HPMC as the main formulation aid resulted in a slow release formulation rather than a rapidly disintegrating one. See Chatlapalli and Rohera, Int. J. Pharmaceutics, 175:47-59 (1998). In addition, waxes, such as glyceryl behenate and glyceryl monostearate, have been used as extrusion aids. See Frisbee, U.S. Pat. No. 6,086,920. However, the use of these waxes as both a binder and filler results in a product that is very sticky and difficult to spheronize. Additionally, pellets containing such high concentrations of wax would be unsuitable for coating purposes.

The initial wet mass formulation prior to extrusion and spheronization should have desired characteristics for both extrusion and spheronization. In other words, the mass should be plastic enough to be extruded through the fine pores of an extruder and should not crumble apart. At the same time, the extrudates obtained from the formulation should be sufficiently brittle so that they may be broken apart and spheronized in a spheronizer. This calls for a balance between the two desired properties i.e. plasticity and brittleness.

At present, the only extrusion-spheronization excipient that has been able to provide this balance is MCC. However, the use of MCC has some disadvantages. Because MCC is water insoluble, pellets made with MCC take a long time to disintegrate and, therefore, release of drug from the pellets is not immediate. For certain applications, such as delayed release dosage forms, this property of MCC may be desirable. However, because MCC-based pellets require some time before the drug releases completely from the pellets, the lack of immediate release from MCC-based pellets would not be desirable for other applications, such as for targeting the drug release locally in the intestine. With MCC-based pellets, limited residence time at the local area in the intestine would therefore riot allow complete absorption of drug.

A significant need therefore exists for a pellet that can be utilized in a multiparticulate dosage form that provides rapid release of a drug from the dosage form, particularly when the dosage form is orally administered.

### BRIEF DESCRIPTION OF THE DRAWINGS

FIG. 1 is a pair of graphs comparing the % drug release from pellets of the invention and from prior art pellets containing MCC when the pellets are situated in phosphate buffer (FIG. 1(a)) or in water (FIG. 1(b)).

### DESCRIPTION OF THE INVENTION

It has been discovered that pellets made by combining a binder with direct compression properties and a waxy spheronizing agent provides characteristics that are desirable for the oral administration of drugs. It has been further discovered that a pellet formulation mass containing a waxy spheronizing agent and a direct compression binder has a desirable balance of plasticity and brittleness and so is useful in manufacturing pellets for oral administration of drugs.

It has been further discovered that dissolving the waxy spheronizing agent in a liquid solvent prior to combining with the other constituents of the formulation mass is of benefit to producing a formulation mass that is to be extruded.

Pellets as described in this application provide an improvement in the release of the drug incorporated in the pellets. The pellets may further provide a pH independent release of the drug, so that the percent drug release is independent of the pH of the gastrointestinal environment.

The direct compression binder is a polymeric material that provides an inherent binding action without the aid of any external agent and is ductile. The direct compression binder should be sufficiently ductile and compressible so that a formulation mass containing the binder may be extruded as an elongated strand. Examples of suitable direct compression binders include but are not limited to various starches such as wheat starch, corn starch, maize starch, and modified starches or starch derivatives such as pre-gelatinized starch and partially pregelatinized starches, microcrystalline starch, microcrystalline cellulose, silicified microcrystalline cellulose, direct compression lactose anhydrous, spray dried lactose, direct compression dicalcium phosphate dihydrate, and direct compression sugars such as mannitol or xylitol. In place of or in combination with a starch, the direct compression binder may include other polymers such as cross-linked polyvinyl pyrrolidone (cross-linked PVP). In a preferred embodiment, the direct compression binder is Starch 1500® (Colorcon Inc., Harleysville, Pa.), a partially pregelatinized maize starch. Preferably, the direct compression binder is present in a concentration range of 2 to 70% of formulation mass and most preferably in the range of 5 to 30% on a non-dried basis.

The waxy spheronizing agent is a wax or a lipid substance that has sufficient plasticity to deform under the pressure used in spheronizing the pellets. The waxy spheronizing agent provides deformability to the pellets and therefore aids in the shaping, such as spheronization, of the pellets produced following extrusion. The waxy spheronizing agent may also provide binding activity, and therefore may also be referred to as a waxy binder/spheronizing agent, although the binding activity of the direct compression binder or of the direct compression binder and a polymeric binder may be sufficient in the absence of the waxy spheronizing agent.

The waxy spheronizing agent, in addition to strengthening the binding action, aids in providing sufficient breakdown and surface smoothness of extrudates during spheronization. This is accomplished as the waxes deform under the moderate pressure conditions present within a spheronizer. Waxy spheronizing agents with melting points of 45° to 50° C. or lower are preferred, although waxes with melting points higher than 50° C. may be used, either alone or in combination with a second waxy spheronizing agent having a melting point of 50° C. or lower.

Waxy spheronizing agents include a wide group of chemicals that includes glycerides, fatty acids, fatty alcohols and their esters, and polyalcohols. Examples of suitable waxy spheronizing agents include polyethylene glycols (PEGs) or derivatives such as fatty acid esters of PEGs, mono, di or tri-glyceryl esters of fatty acids such as glyceryl stearate, glyceryl oleate, glyceryl behenate, and glyceryl palmitate-stearate, polyethyleneglycol glycerides such as sold under the tradename GELUCIRE® (Gattefosse, Saint-Priest, France). Preferred waxy spheronizing agents include Gelucire® 50/13 and Gelucire® 44/14. Gelucire® 50/13 is composed of composed of fatty acid (C16 and C18) esters of glycerol, polyethylene glycol (PEG) esters and free PEG. Gelucire® 44/14 is composed of polyethylene glycol 33, PEG mono-and diesters of fatty acids, glycerides, and glycerol. The waxy spheronizing agent is generally present in the concentration range of 1 to 50% of formulation mass and most preferably in the range of 1 to 25%.

The pellet formulation mass is made by combining the direct compression binder and the waxy spheronizing agent in a suitable wetting liquid, such as a hydrophilic liquid. Preferably, the hydrophilic liquid is water. One or more organic solvents may be utilized in addition to water or in place of water. Such organic solvents are not preferred, however, because of the desirability of removal of such solvents from the formulation mass during the pelleting process. In contrast, excess water remaining in the pellets typically does not present a problem.

Additional ingredients may be included in the mass, if desired. Such additional ingredients may include any ingredients that are used in solid pharmaceutical dosage forms, such as binders like polymeric binders, fillers like lactose or dicalcium phosphate dihydrate, or disintegrants such as cross-carmellose sodium, sodium starch glycolate, or cross-povidone.

The polymeric binder, if present, can be a natural binder or a synthetic/semisynthetic binder. Natural polymeric binders include but are not limited to acacia, tragacanth, gelatin, starch paste, alginic acid and cellulose. Synthetic/semisynthetic polymeric binders include but are not limited to methyl cellulose, ethyl cellulose, hydroxypropyl methyl cellulose, hydroxypropyl cellulose, sodium carboxymethyl cellulose, polyvinyl pyrrolidone, polyethylene glycol, polyvinyl alcohol and polymethacrylates. The polymeric binder preferably is a cellulosic polymer such as hydroxypropyl-methyl cellulose (HPMC), methyl cellulose (MC), hydroxypropyl cellulose (HPC), or hydroxyethyl cellulose (HEC). Preferably, the cellulosic polymer binder is other than microcrystalline cellulose. If desired, the cellulosic binder may be a combination of microcrystalline cellulose and another cellulosic polymer.

Polymeric binders other than those based on cellulose may be used. The polymeric binder should be sufficiently ductile and compressible so that a formulation mass containing the binder may be extruded as an elongated strand. Examples of polymeric binders other than cellulosic polymers that are suitable include polyvinyl pyrrolidone (PVP) and PVP copolymers.

Polymeric binders are generally used in the concentration range of 0 to 10% of the formulation mass, most preferably in the range of 2 to 5%. Lower concentrations are preferred as higher levels of polymeric binder tend to result in sticky pellets or even a slower drug release.

In a preferred embodiment, the direct compression binder is combined with the waxy spheronizing agent. If desired, the additional ingredients, such as a polymeric binder, may be combined with the direct compression binder. The waxy spheronizing agent may also be combined with the direct compression binder, either as a powder or in a melted or dissolved form. However, in a preferred embodiment if the waxy spheronizing agent is hydrophilic, such as a PEG or a Gelucire, the waxy spheronizing agent is dissolved in a solvent such as a hydrophilic solvent like water prior to being combined with the other ingredients and the dissolved waxy spheronizing agent is then added to the direct compression binder.

Preferably, the waxy spheronizing agent is added to the mixture gradually. If the waxy spheronizing agent is added too quickly, the mixture may produce lumps. If the waxy spheronizing agent is added too slowly, the mixture may become too dense. Therefore, the rate of addition of the dissolved waxy spheronizing agent is adjusted to produce a mixture with the desired physical characteristics. If the waxy spheronizing agent is hydrophobic, such as a glycerol ester of a fatty acid, it is preferred that the waxy spheronizing agent be dispersed as a fine powder with the direct compression binder. Generally, if a hydrophobic binder is utilized, it is preferred to utilize also a hydrophilic binder, which is preferably dissolved in a solvent prior to combining with the other ingredients.

In a most preferred embodiment, the formulation mass is made as follows: All of the ingredients except the waxy spheronizing agent are combined and mixed, such as in a Robot Coupe mixer (Robot Coupe USA, Inc., Jackson, Miss.) with the blades in reverse direction for 3 minutes to obtain a powder blend. The required amount of the waxy spheronizing agent, such as Gelucire®, is dissolved, such as in water. Heating may be utilized to aid in the dissolution of the waxy spheronizing agent. The waxy spheronizing agent solution is then added, preferably gradually, to the powder blend.

A drug may be combined with the other ingredients to provide a drug-containing formulation mass. The drug may be combined with the direct compression binder followed by the addition of the waxy spheronizing agent or may be combined following the combination of the direct compression binder and waxy spheronizing agent. The drug may be a hydrophilic drug, such as acetaminophen, or may be a hydrophobic drug such as furosemide.

The formulation mass may then be transferred to an extruder for extrusion. For example, the material may be extruded using a 0.7 size dome shaped assembly at a speed of 50 rpm. If desired, the material may be re-extruded. The extrudates are then transferred to a spheronizer for spheronization. For example, spheronization may be at 1600 to 1800 rpm for 10-15 minutes. Following spheronization, the obtained pellets are dried, such as in a fluid bed dryer at a temperature of 50° C. for a period of less or more than 20 minutes.

The pellets may be compressed into a unitary solid form to provide tablets or may be loaded as a multiplicity of discrete pellets into shells, such as a gelatin or HPMC capsule or shell, to form capsules. Such tablets or capsules may be utilized for oral administration of drugs. The pellets may further be coated with either functional or non-functional coatings.

The pellets made in accordance with this application provide the unexpected advantageous property that the pellets rapidly disintegrate and so provide fast release of drugs, including hydrophilic and hydrophobic drugs. The invention is further described in the following examples that are provided for the purpose of illustration and not limitation and to disclose examples of preparation of representative forms of embodiments of this invention. It is noted that, in the examples, certain components are utilized in the formation of the pellets. Such components are provided as illustrations and not as limitations. For example, starch is utilized in the examples as an illustration of a direct compression binder and Gelucire® 50/13 is utilized as an illustration of a waxy spheronizing agent.

### EXAMPLE 1

Pellets were prepared containing the ingredients as indicated below in Table 1.

TABLE 1

| | Percent by weight of |
|---|---|
| Ingredient | composition |
| Acetaminophen | 29.90 |
| Lactose monohydrate | 23.36 |
| Starch 1500 | 23.36 |
| Gelucire ® 50/13 | 9.34 |
| Hydroxypropyl methylcellulose (HPMC E15 LV) | 2.80 |
| Purified water | 11.21 |

The hydroxypropyl methylcellulose, lactose monohydrate, starch 1500, and acetaminophen were blended in a high shear mixer (Robot Coupe Model: 3VG) for 2 minutes with the blades rotating in reverse direction to form a powder bed. A waxy spheronizing agent in liquid form (Gelucire mixed with water) was added to the powder bed with the mixer blades rotating in the forward direction until the entire liquid was consumed. Mixing was stopped and the wet formulation mass obtained was transferred to an extruder (Multigranulator®, Model: MG-55, LCI Corp, Charlotte, N.C.) which was set at 50 rpm. The extrudates were then conveyed to a spheronizer (Benchtop Marumerizer®, Model: QJ-230-T-1, LCI Corp) rotating at 1500 rpm for 20 minutes. The pellets obtained were dried in a mini fluid bed dryer (Aeromatic-Fielder™, GEA Pharma Systems, Belgium) at 50° C. for 15 minutes.

The pellet aspect ratio (A_{R}) was determined by using ImageJ software (http://rsbweb.nih.gov/ij/) and measurement of the largest Feret's diameter (dₘₐₓ) and the smallest Feret's diameter (dₘᵢₙ) of the pellets and calculated by the formula: A_{R}=dₘₐₓ/dₘᵢₙ. For the pellets in Table 1, the aspect ratio was determined to be 1.23. The pellet disintegration time was determined by immersing the pellets in water, allowing the pellets to remain undisturbed in the water, and determining by visual observation of the time required for the pellets to divide into numerous pieces and lose their shape as defined pellets. For the pellets in Table 1, the disintegration time was determined to be less than 5 minutes.

### EXAMPLE 2

Pellets were prepared containing the ingredients as indicated below in Table 2. The pellets were prepared as in Example 1 with the replacement of the active pharmaceutical ingredient furosemide in place of acetaminophen.

TABLE 2

| Ingredient | Percent by weight of composition |
|---|---|
| Furosemide | 29.90 |
| Lactose monohydrate | 35.05 |
| Starch 1500 | 11.68 |
| Gelucire ® 50/13 | 9.34 |
| HPMC E15 LV | 2.80 |
| Purified water | 11.21 |

For the pellets in Table 2, the aspect ratio was determined to be 1.19 and the disintegration time was determined to be less than 5 minutes.

### EXAMPLE 3

Three batches were prepared with different compositions in order to characterize the individual effects of the inclusion or omission of the individual direct compression binder and waxy spheronizing agent on pellet properties. The list of ingredients for the three batches is listed in Table 3. The pellets were prepared as described in Example 1 with the exception of the inclusion or omission of particular ingredients as indicated in Table 3.

TABLE 3

| | Percent by weight of composition | | |
|---|---|---|---|
| Ingredient | Batch I | Batch II | Batch III |
| Acetaminophen | 29.90 | 29.90 | 29.90 |
| Lactose monohydrate | 56.07 | 46.73 | 35.05 |
| Starch 1500 | — | — | 11.68 |
| Gelucire ® 50/13 | — | 9.34 | 9.34 |
| HPMC E15 LV | 2.80 | 2.80 | 2.80 |
| Purified water | 11.21 | 11.21 | 11.21 |

The aspect ratios for the three batches were 1.42 for Batch I, 1.11 for Batch II, and 1.18 for Batch III, which indicates an improved sphericity with the presence of the waxy spheronizing agent. Additionally, the combination of the direct compression binder and the waxy spheronizing agent provided an improved pellet size distribution, as shown below in Table 4.

TABLE 4

| Size (mm) | Batch I | Batch II | Batch III |
|---|---|---|---|
| 2 | 0 | 0 | 0 |
| 0.841 | 64.07767 | 59.2233 | 96.60194 |
| 0.707 | 27.6699 | 26.21359 | 5.339806 |
| 0.42 | 23.78641 | 13.59223 | 0.485437 |
| 0.25 | 5.339806 | 1.456311 | 0.485437 |
| 0.177 | 0.485437 | 0.485437 | 0 |
| 0.125 | 0 | 0 | 0.485437 |

### EXAMPLE 4

Pellets were prepared containing the ingredients listed in Table 5.

TABLE 5

| Ingredient | Percent by weight of composition |
|---|---|
| Starch | 6.277805 |
| Microcrystalline starch | 6.277805 |
| Lactose monohydrate | 13.02521 |
| Cross carmellose sodium | 5.066733 |
| Silicon dioxide (Cab-o-sil) | 1.235788 |
| Starch 1500 | 10.77608 |
| Gelucire ® 50/13 | 4.201681 |
| Wheat flour | 4.943154 |
| Glyceryl monostearate | 1.235788 |
| HPMC E15 LV | 0.741473 |
| Sodium starch glycolate | 1.235788 |
| Water | 44.9827 |

All ingredients except Gelucire and water were dry mixed in a high shear mixer (Robot Coupe Model: 3VG) for 3 minutes with the blades rotating in reverse direction to form a powder bed. A waxy spheronizing agent in liquid form (Gelucire and water) was then added to the powder bed with the mixer blades rotating in the forward direction until the entire liquid was consumed. Mixing was stopped and the wet mass obtained was transferred to the extruder (Multigranulator®, Model: MG-55, LCI Corp, Charlotte, N.C.) which was set at 50 rpm. The extrudates were then conveyed to the spheronizer (Benchtop Marumerizer®, Model: QJ-230-T-1, LCI Corp) rotating at 1600 rpm for 20 minutes. The pellets obtained were dried in a mini fluid bed dryer (Aeromatic-Fielder™, GEA Pharma Systems, Belgium) at 50° C. for 20 minutes.

For the pellets in Table 5, the aspect ratio was determined to be 1.14 and the disintegration time was determined to be less than 2 minutes.

### EXAMPLE 5

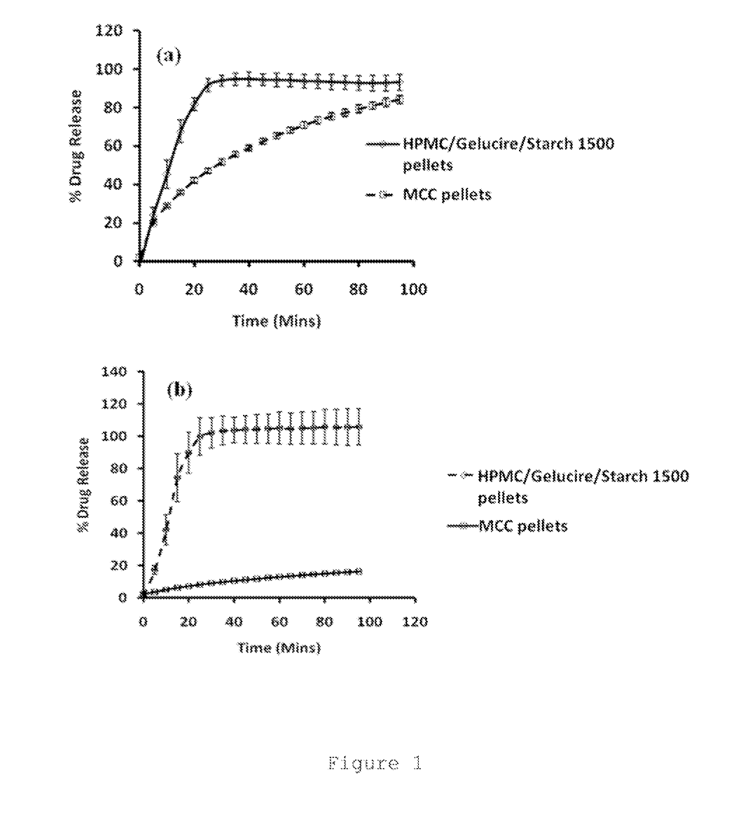

The pellets containing the hydrophobic drug furosemide of Example 2 and similar pellets made with MCC in place of lactose monohydrate, starch, and Gelucire were analyzed to determine the rate of release of the drug from the pellets. The pellets were placed in dissolution medium of either (a) pH 7.4 phosphate buffer or (b) water. The dissolution medium was maintained at a temperature of 37° C.±0.5° C. in a USP Type 1 dissolution basket apparatus at 75 rpm. The drug concentration was determined by an in line UV spectrophotometer using a fiber optic probe. The results are shown diagrammatically in FIGS. 1(a) and 1(b).

As shown in FIG. 1(a), when the pellets were dissolved in PBS, release of drug from the pellets of the present application occurred rapidly with almost 100% of drug released within 20 minutes. In contrast, drug release from MCC-based pellets proceeded much more slowly, with about 80% of drug released within 80 minutes.

As shown in FIG. 1(b), when the pellets were dissolved in water, release of drug from the pellets of the present application likewise occurred rapidly, with about 100% of drug released within 20 to 30 minutes. In contrast, drug release from MCC-based pellets proceeded very slowly, with only about 10% of drug released within 100 minutes.

The pellets of the present application and prior art MCC-based pellets were compared visually after having been in water for 90 minutes. The pellets of the present application had disintegrated, were no longer visible, and had been completely dissolved. The MCC-based pellets were still visible and had retained their shape and structure.

### EXAMPLE 6

Pellets were made according to Example 1 either (a) without a waxy spheronizing agent or a direct compression binder, (b) with a waxy spheronizing agent (Gelucire® 50/13) and without a direct compression binder, and (c) with the waxy spheronizing agent and a direct compression binder (Starch 1500). Similar pellets lacking these ingredients but containing MCC were made.

The pellets were tested for friability by loading an accurately weighed amount of the pellets into a glass cylindrical container with fine mesh on both sides. The cylinder was rotated for 4 minutes at 25 rpm using a Vanderkamp Sustained Release apparatus (Agilent Technologies, Santa Clara, Calif.). Pellets retained on US sieve #40 were used for this test. Around 10 g of pellets were weighed and loaded into the cylindrical glass container along with 10 g of glass beads. After completion of the test, the pellets were subjected to sieving from a US sieve #40 to separate any fined generated during the test. The pellets retained on the sieve were then weighed. The percent friability was calculated as follows and is shown in Table 6.

% Friability=(Initial weight−Final weight)*100/Initial weight

TABLE 6

| Pellets | % Friability |
|---|---|
| No waxy spheronizing agent or direct | 0.192567 |
| compression binder | |
| Waxy spheronizing agent but no direct | 0.288385 |
| compression binder | |
| Waxy spheronizing agent and direct | 0.001882 |
| compression binder | |
| MCC | <0.01 |

As shown in Table 6, pellets containing a waxy spheronizing agent and a direct compression binder exhibited greatly reduced friability compared to pellets lacking either or both of the waxy spheronizing agent and the direct compression binder. The pellets containing the waxy spheronizing agent and the direct compression binder exhibited friability comparable or even less than that of pellets containing MCC.

The pellets of the present application provide several advantages over those of the prior art. Unlike prior art pellets containing MCC, the pellets of the present application disintegrate rapidly in aqueous fluids and rapidly release drug contained in the pellets, including both hydrophilic and hydrophobic drugs. The ability of the pellets of the present application to release drug rapidly is extremely advantageous in situations where it is desirable for the drug to be released almost immediately once the pellets reach the desired site. One such instance is when delivering drug to the intestinal tract, particularly to the colon. Due to the limited residence time of a dosage form, such as pellets, at the target site, rapid release of drug is desirable. Such rapid release, which is provided by the pellets of the present application, was not obtainable with prior art pellets.

Various modifications of the above described invention will be evident to those skilled in the art. It is intended that such modifications are included within the scope of the following claims.

1. A formulation mass for making pellets for oral administration comprising a direct compression binder and a waxy spheronizing agent.

2. The formulation mass of claim 1 that further comprises a polymeric binder.

3. The formulation mass of claim 1 that further comprises a wetting liquid.

4. The formulation mass of claim 1 wherein the direct compression binder is a starch or a starch derivative.

5. The formulation mass of claim 4 wherein the starch or starch derivative is a partially pregelatinized maize starch.

6. The formulation mass of claim 1 wherein the waxy spheronizing agent is a polyethyleneglycol glyceride.

7. The formulation mass of claim 1 which further comprises an active pharmaceutical ingredient.

8. A method for making a pellet comprising compressing the formulation mass of claim 1.

9. A method for making a pellet comprising compressing the formulation mass of claim 7.

10. A pellet made by the method of claim 8.

11. The pellet of claim 10 which comprises a coating.

12. A pellet made by the method of claim 9.

13. A tablet for oral administration comprising a multiplicity of the pellets of claim 10 that are compressed into a solid form.

14. A capsule for oral administration comprising a multiplicity of the pellets of claim 10 that are placed within a capsule shell.

15. A method for making a formulation mass for making pellets comprising adding a waxy spheronizing agent to a direct compression binder.

16. The method of claim 15 wherein the waxy spheronizing agent is dissolved in a liquid solvent.

17. The method of claim 15 wherein the direct compression binder is combined with a polymeric binder.

18. A method for making pellets comprising extruding the formulation mass of claim 1 into strands, breaking the strands into a multiplicity of pieces, and spheronizing the pieces to yield pellets.

19. The method of claim 18 which further comprises coating the pellets with a functional or non-functional coating.

20. A method for making a tablet for oral administration comprising compressing the pellets obtained by the method of claim 18 into the form of a tablet.

21. A method for making a capsule for oral administration comprising loading a multiplicity of the pellets obtained by the method of claim 18 into a capsule shell.

22. The formulation mass of claim 1 wherein the direct compression binder is a partially pregelatinized starch and the waxy spheronizing agent is a polyethyleneglycol glyceride.

23. The formulation mass of claim 2 wherein the direct compression binder is a partially pregelatinized starch and the waxy spheronizing agent is a polyethyleneglycol glyceride.

24. The formulation mass of claim 23 wherein the polymeric binder is hydroxypropyl methylcellulose.
